(19) United States
(12) Patent Application Publication
(10) Pub. No.: US 2024/0238178 A1
(43) Pub. Date: Jul. 18, 2024

(54) COMPOSITION COMPRISING AHAS AND/OR BHAS WHICH IS STABILIZED BY A COMBINATION OF MONOALKYL OR DIALKYL ISOSORBIDE, POLYETHYLENE GLYCOL-POLYPROPYLENE GLYCOL ETHER AND TWO HYDROGENATED OR NON-HYDROGENATED POLYOXYETHYLENATED PLANT OILS

(71) Applicant: L'OREAL, Paris (FR)

(72) Inventors: Sabina MERALLI-BALLOU, CHEVILLY LARUE (FR); Julie CRIBIER, CHEVILLY LARUE (FR)

(73) Assignee: L'OREAL, Paris (FR)

(21) Appl. No.: 18/559,805

(22) PCT Filed: Jun. 27, 2022
(86) PCT No.: PCT/EP2022/067582

§ 371 Date: Nov. 9, 2023

(30) Foreign Application Priority Data
Jun. 28, 2021 (FR) FR2106942

(51) Int. Cl. A61K 8/365; A61K 8/34; A61K 8/368; A61K 8/37; A61K 8/49; A61K 8/92; A61Q 19/10

(52) U.S. Cl. CPC A61K 8/365; A61K 8/345; A61K 8/368; A61K 8/375; A61K 8/4973; A61K 8/922; A61Q 19/10

(57) ABSTRACT

The present invention relates to a composition, preferably a cosmetic or dermatological composition, comprising:
i. at least one hydroxy acid chosen from α-hydroxy acids, β-hydroxy acids, and mixtures thereof;
ii. at least one monoalkyl or dialkyl isosorbide, the alkyl group comprising from 1 to 5 carbon atoms;
iii. at least one polyethylene glycol-polypropylene glycol ether of formula (I): CₙH₂ₙ₊₁(OCH(CH₃)CH₂)ₓ(OCH₂CH₂)_{y}OH (I);
iv. at least one first hydrogenated or non-hydrogenated, polyoxyethylenated plant oil and at least one second hydrogenated or non-hydrogenated, polyoxyethylenated plant oil; said first and second polyoxyethylenated plant oils being different from one another;
v. a physiologically acceptable medium comprising at least water;
vi. said composition having an acidic pH.

The present invention also relates to a cosmetic treatment method comprising the application of said composition to keratin materials and also to the cosmetic use of said composition for caring for the skin.

The present invention relates to the field of cosmetic or dermatological compositions for caring for keratin materials, notably the skin. The present invention relates to a cosmetic or dermatological composition comprising AHAs and/or BHAs which is stabilized by a combination of monoalkyl or dialkyl isosorbide, polyethylene glycol-polypropylene glycol ether and two hydrogenated or non-hydrogenated polyoxyethylenated plant oils, to a cosmetic treatment method comprising a step of applying said composition to keratin materials, preferably the skin, and also to the use thereof for caring for keratin materials, preferably the skin.

It is known practice to use, as active agents in the cosmetic or dermatological compositions, acidic agents such as hydroxy acids. These active agents are notably used to care for the face and/or body and more especially to give the face a luminous and radiant complexion and therefore a healthy glow, a smooth and younger appearance, to treat skin wrinkles and fine lines, to depigment the skin and notably to remove the marks that appear with aging, and also to eliminate comedones.

The compositions conventionally used in the cosmetic or dermatological fields are water-in-oil (W/O) emulsions, oil-in-water (O/W) emulsions, or aqueous gels, into which it is often difficult, or even impossible, to incorporate organic acidic active agents since these have a tendency to destabilize the compositions containing them.

Specifically, these acidic active agents generally have a tendency to recrystallize or to degrade. This results in greater or lesser loss of efficacy of these compositions, depending on the degree of recrystallization and/or degradation, which runs counter to the desired objective. Moreover, this recrystallization or degradation may modify the overall stability of these compositions and also the appearance and consistency thereof (loss of viscosity and fluidization), which may turn the user away from these specific treatment compositions.

The need thus remains for a stable composition that is in the form of an aqueous gel or an emulsion, which can be used in particular in cosmetic and/or dermatological fields and which enables a sufficient incorporation of the acidic active agents generally used in these fields for the purpose of maximum efficacy, while retaining a good stability.

The applicant has surprisingly discovered that a combination of monoalkyl or dialkyl isosorbide, polyethylene glycol-polypropylene glycol ether of formula (I) below and hydrogenated or non-hydrogenated polyoxyethylenated plant oils makes it possible to obtain cosmetic or dermatological compositions comprising one or more hydroxy acids that are stable over time at room temperature and also at temperatures of 4° C. or 45° ° C. for 2 months.

The composition according to the invention notably has, besides the abovementioned good stability properties, good sensory properties, in particular the absence of a tacky sensation after application.

Thus, a first subject of the present invention is a cosmetic or dermatological composition, comprising:
i. at least one hydroxy acid chosen from α-hydroxy acids, β-hydroxy acids, and mixtures thereof;
ii. at least one monoalkyl or dialkyl isosorbide, the alkyl group comprising from 1 to 5 carbon atoms;
iii. at least one polyethylene glycol-polypropylene glycol ether of formula (I):

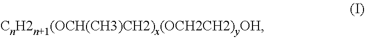

in which:
   a. n is an integer ranging from 1 to 24, preferably ranging from 4 to 22, better still ranging from 4 to 18, even more preferentially ranging from 4 to 12, such as 4;
   b. x is an integer ranging from 1 to 40, preferably ranging from 2 to 30, better still ranging from 10 to 30, such as 26;
   c. y is an integer ranging from 1 to 40, preferably ranging from 2 to 30, better still ranging from 10 to 30, such as 26;
iv. at least one first hydrogenated or non-hydrogenated, polyoxyethylenated plant oil and at least one second hydrogenated or non-hydrogenated, polyoxyethylenated plant oil; said first and second polyoxyethylenated plant oils being different from one another;
V. a physiologically acceptable medium comprising at least water;
vi. said composition having an acidic pH.

Another subject of the present invention is a cosmetic treatment method for keratin materials, preferably the skin, comprising a step of applying said composition to said keratin materials, preferably the skin.

The invention also relates to the cosmetic use of said composition for caring for keratin materials, preferably the skin.

### Definition

The term “physiologically acceptable medium” is understood in the present application to mean a medium that is compatible with all keratin materials such as the skin, including the scalp, the nails, mucous membranes, the eyes and the hair, or any other area of bodily skin.

Said physiologically acceptable medium comprises water and optionally one or more water-miscible organic solvents which may be chosen from linear or branched C1-C6 monoalcohols such as ethanol, isopropanol or tert-butanol; polyols such as glycerol, propylene glycol, hexylene glycol (or 2-methyl-2,4-pentanediol), and polyethylene glycols; polyol ethers such as dipropylene glycol monomethyl ether; and mixtures thereof.

Preferentially, the composition according to the invention has a water content ranging from 20% to 95% by weight, better still from 40% to 90% by weight, relative to the total weight of the composition.

Advantageously, the composition according to the invention comprises one or more water-miscible organic solvents in a content ranging from 0.5% to 25% by weight, preferably from 5% to 20% by weight.

An “acidic pH” is understood to mean a pH of less than 6, preferably equal to or less than 5, better still chosen from the range of from 2 to 5, such as 3.8. Above the value of pH 6, the formulation of acidic active agents no longer presents difficulties since the acid is in general completely neutralized.

The expression “good stability properties” or “stable composition” is understood to mean a clear and/or borderless composition, exhibiting no recrystallization phenomenon at room temperature (25° C.), and/or at 4° C. and/or at 45° C. for a period of 2 months.

### DETAILED DESCRIPTION OF THE INVENTION

### Composition

A first subject of the present invention is a cosmetic or dermatological composition, comprising:
i. at least one hydroxy acid chosen from α-hydroxy acids, β-hydroxy acids, and mixtures thereof;
ii. at least one monoalkyl or dialkyl isosorbide, the alkyl group comprising from 1 to 5 carbon atoms;
iii. at least one polyethylene glycol-polypropylene glycol ether of formula (I):

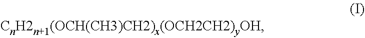

in which:
   a. n is an integer ranging from 1 to 24, preferably ranging from 4 to 22, better still ranging from 4 to 18, even more preferentially ranging from 4 to 12, such as 4;
   b. x is an integer ranging from 1 to 40, preferably ranging from 2 to 30, better still ranging from 10 to 30, such as 26;
   c. y is an integer ranging from 1 to 40, preferably ranging from 2 to 30, better still ranging from 10 to 30, such as 26;
iv. at least one first hydrogenated or non-hydrogenated, polyoxyethylenated plant oil and at least one second hydrogenated or non-hydrogenated, polyoxyethylenated plant oil; said first and second polyoxyethylenated plant oils being different from one another;
V. a physiologically acceptable medium comprising at least water;
vi. said composition having an acidic pH.

### Hydroxy Acids

Advantageously, the α-hydroxy acids are chosen from glycolic acid, citric acid, lactic acid, tartaric acid, malic acid, mandelic acid, and mixtures thereof, better still the α-hydroxy acids are chosen from citric acid, glycolic acid and mixtures thereof.

Advantageously, the β-hydroxy acids are chosen from salicylic acid and the salicylic acid derivatives of the following formula (II):

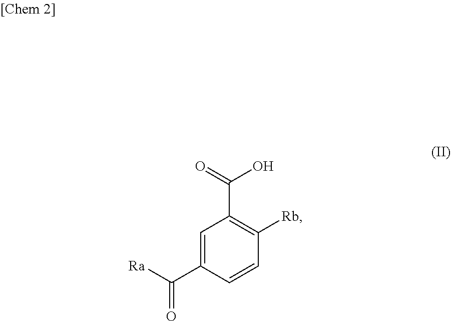

wherein:
i. the radical Ra denotes:
   a. a linear, branched or cyclic, saturated aliphatic chain containing from 2 to 22 carbon atoms;
   b. an unsaturated chain containing from 2 to 22 carbon atoms, containing one or more double bonds which may be conjugated;
   c. an aromatic nucleus bonded to the carbonyl radical directly or by means of saturated or unsaturated aliphatic chains containing from 2 to 7 carbon atoms;
      it being possible for said groups to be substituted with one or more identical or different substituents chosen from: (a) halogen atoms, (b) the trifluoromethyl group, (c) hydroxyl groups in free form or esterified with an acid containing from 1 to 6 carbon atoms, or (d) a carboxyl function in free form or esterified with a lower alcohol containing from 1 to 6 carbon atoms;
ii. Rb is a hydroxyl group;
   and also the salts thereof derived from a mineral or organic base and mixtures thereof.

According to one embodiment, the radical Ra denotes:
a linear, branched or cyclic, saturated aliphatic chain containing from 3 to 11 carbon atoms; or
an unsaturated chain containing from 3 to 17 carbon atoms and comprising one or more conjugated or non-conjugated double bonds;
it being possible for said hydrocarbon chains to be substituted with one or more substituents, which may be identical or different, chosen from: (a) halogen atoms; (b) the trifluoromethyl group; (c) hydroxyl groups in free form or esterified with an acid containing from 1 to 6 carbon atoms; and (d) a carboxyl function in free form or esterified with a lower alcohol containing from 1 to 6 carbon atoms;
and also the salts thereof obtained by salification with a mineral or organic base.

The compounds that are more particularly preferred are those in which the radical Ra is a C3-C11 alkyl group. Among the compounds of formula (II) that are particularly preferred, mention may be made of: 5-n-octanoylsalicylic acid (or capryloylsalicylic acid); 5-n-decanoylsalicylic acid; 5-n-dodecanoylsalicylic acid; 5-n-heptyloxysalicylic acid, and the corresponding salts thereof.

The salicylic acid compound is advantageously chosen from salicylic acid and 5-n-octanoylsalicylic acid. 5-n-Octanoylsalicylic acid will more particularly be used. 5-n-Octanoylsalicyliic acid (or capryloylsalicylic acid) is provided under the name Mexoryl SAB® by Chimex.

Examples of mineral bases that may be mentioned include alkali metal or alkaline-earth metal hydroxides, for instance sodium hydroxide, potassium hydroxide or ammonium hydroxide. Among the organic bases, mention may be made of amines and alkanolamines.

Quaternary salts, for instance those described in the patent FR 2 607 498, are particularly advantageous. The compounds of formula (II) that can be used according to the invention are described in patents U.S. Pat. Nos. 6,159,479, 5,558,871, FR 2 581 542, FR 2 607 498, U.S. Pat. No. 4,767,750, EP 378 936, U.S. Pat. Nos. 5,267,407, 5,667,789, 5,580,549 and EP A-570 230.

The hydroxy acid(s), α-hydroxy acid(s) and/or β-hydroxy acid(s), may be present in the composition according to the invention in an amount ranging from 2% to 20% by weight relative to the total weight of the composition, preferably ranging from 4% to 13% by weight relative to the total weight of the composition, better still ranging from 5% to 10% by weight relative to the total weight of the composition.

In one preferred embodiment, the hydroxy acids are present in the form of a mixture of α-hydroxy acid(s) and β-hydroxy acid(s). More preferably still, the mixture of α-hydroxy acid(s) and β-hydroxy acid(s) comprises:
at least one α-hydroxy acid present in the composition according to the invention in an amount ranging from 2% to 15% by weight, relative to the total weight of the composition, preferably ranging from 3% to 10% by weight relative to the total weight of the composition, better still ranging from 4% to 7% by weight relative to the total weight of the composition, for example 4.5% by weight relative to the total weight of the composition;
at least one β-hydroxy acid present in the composition according to the invention in an amount ranging from 0.1% to 5% by weight, relative to the total weight of the composition, preferably ranging from 1% to 3% by weight relative to the total weight of the composition, for example 2% by weight relative to the total weight of the composition.

### Monoalkyl or Dialkyl Isosorbide

Advantageously, the one monoalkyl or dialkyl isosorbide is dimethyl isosorbide. Mention may in particular be made of the dimethyl isosorbide sold under the trade name ARLASOLVE DMI-PC-LQ-(AP) by CRODA or else DOTTISOL 08820.018 sold by DOTTIKON EXCLUSIVE SYNTHESIS.

Said monoalkyl or dialkyl isosorbide may be present in the composition according to the invention in an amount ranging from 0.5% to 10% by weight, relative to the total weight of the composition, preferably ranging from 1% to 5% by weight relative to the total weight of the composition, for example 2% by weight relative to the total weight of the composition.

### Polyethylene Glycol-Polypropylene Glycol Ether

Advantageously, said polyethylene glycol-polypropylene glycol ether of formula I is chosen from cetyl alcohol oxyethylenated with 20 oxyethylene units and oxypropylenated with 5 oxypropylene units such as the product sold under the name PROCETYL AWS® by Croda, butyl alcohol oxyethylenated with 26 oxyethylene units and oxypropylenated with 26 oxypropylene units such as the product sold under the name PPG-26-BUTETH-26® by Goldschmidt, decyltetradecanol oxyethylenated with 30 oxyethylene units and oxypropylenated with 6 oxypropylene units such as the product sold under the name NIKKOL PEN-4630® by Nikkol, lauryl alcohol oxyethylenated with 25 oxyethylene units and oxypropylenated with 25 oxypropylene units such as the product sold under the name ADF-OLEILE® by Vevy, and mixtures thereof, better still, butyl alcohol oxyethylenated with 26 oxyethylene units and oxypropylenated with 26 oxypropylene units.

Said polyethylene glycol-polypropylene glycol ether of formula I may be present in the composition according to the invention in an amount ranging from 0.1% to 5% by weight, relative to the total weight of the composition, preferably ranging from 0.5% to 2% by weight relative to the total weight of the composition, for example 1% by weight relative to the total weight of the composition.

Two hydrogenated or non-hydrogenated polyoxyethylenated plant oils

Advantageously, the first hydrogenated or non-hydrogenated polyoxyethylenated plant oil comprises from 2 to 50 oxyethylene units, preferably from 30 to 50 oxyethylene units, such as 40 oxyethylene units.

Better still, the first polyoxyethylenated plant oil is a hydrogenated or non-hydrogenated polyoxyethylenated castor oil comprising from 2 to 50 oxyethylene units, preferably from 30 to 50 oxyethylene units.

More preferentially still, the first polyoxyethylenated plant oil is a hydrogenated polyoxyethylenated castor oil comprising 40 oxyethylene units such as that sold under the name SOLUBILISANT LRI by SENSIENT.

Said first hydrogenated or non-hydrogenated polyoxyethylenated plant oil may be present in the composition according to the invention in an amount ranging from 0.1% to 2% by weight, relative to the total weight of the composition, preferably ranging from 0.5% to 0.8% by weight relative to the total weight of the composition, for example 0.7% by weight relative to the total weight of the composition.

Advantageously, the second hydrogenated or non-hydrogenated polyoxyethylenated plant oil comprises from 51 to 100 oxyethylene units, preferably from 55 to 70 oxyethylene units, such as 60 oxyethylene units.

Better still, the second polyoxyethylenated plant oil is a hydrogenated or non-hydrogenated polyoxyethylenated castor oil comprising from 51 to 100 oxyethylene units, preferably from 55 to 70 oxyethylene units.

More preferentially still, the second polyoxyethylenated plant oil is a hydrogenated polyoxyethylenated castor oil comprising 60 oxyethylene units such as that sold under the name EMULSOGEN HCO 060 by CLARIANT.

Said second hydrogenated or non-hydrogenated polyoxyethylenated plant oil may be present in the composition according to the invention in an amount ranging from 0.05% to 0.5% by weight, relative to the total weight of the composition, preferably ranging from 0.1% to 0.4% by weight relative to the total weight of the composition, for example 0.3% by weight relative to the total weight of the composition.

### Polysaccharide

The composition according to the invention may further comprise at least one polysaccharide.

A polysaccharide used according to the present invention may, in addition, or alternatively, be in linear or branched form.

Within the meaning of the present invention, the term “branched” denotes a polymer of which at least one saccharide unit and/or one polysaccharide block, is attached laterally to the main chain.

In the context of the present invention, the terms “radical” and “unit” will be used interchangeably to denote a monosaccharide unit or derivative, forming a polysaccharide used according to the invention.

As saccharide units or radicals, mention may for example be made, besides fucose, of glucose and glucuronic acid, galacturonic acid, trioses such as ketotrioses (dihydroxyacetone), aldotrioses (glyceraldehyde); tetroses such as ketotetroses (erythrulose), aldotetrose (erythrose, threose); pentoses such as ketopentoses (ribulose, xylulose), aldopentose (arabinose, lyxose, ribose, xylose), deoxy sugar (deoxyribose); hexoses such as ketohexoses (fructose, psicose, sorbose, tagatose), aldohexoses (allose, altrose, galactose, gulose, idose, mannose, talose), deoxy sugars (fuculose, pneumose, quinovose, rhamnose); heptoses (glucoheptose, idoheptulose, mannoheptulose, sedoheptulose, taloheptulose) or else octoses or derivatives thereof.

The derivatives of a saccharide unit are understood to mean a unit as defined above that has additionally undergone a chemical modification resulting in the presence of additional chemical groups and/or in the absence of chemical groups initially present within the monosaccharide under consideration. By way of example, mention may for example be made of galacturonic acid, derived from galactose, oxidized at carbon number 6 thereof.

Advantageously, the saccharide units used within a saccharide polymer according to the invention may form a saccharide block which is repeated within said polymer.

Preferably, the polysaccharide(s) is (are) present in the composition according to the invention in an amount ranging from 0.1% to 2% by weight, relative to the total weight of the composition, preferably ranging from 0.2% to 1% by weight relative to the total weight of the composition.

In a first embodiment, a polysaccharide used according to the present invention is an anionic polysaccharide containing fucose, galactose and galacturonic acid.

Preferentially, a polysaccharide used according to the present invention is an anionic polysaccharide comprising at least three saccharide units or derivatives thereof, including at least one of fucose type, one of galactose type and one of galacturonic acid type, these units being bonded together via O-glycoside bonds.

More preferentially, a polysaccharide used according to the present invention is an anionic polysaccharide containing L-fucose, D-galactose and galacturonic acid.

Better still, a saccharide polymer that is very particularly suitable according to the present invention is formed solely of fucose, galactose and galacturonic acid units.

Such a polymer is notably sold under the name Fucogel Powder® (INCI name: Biosaccharide Gum-1), sold by SOLABIA.

In a second embodiment, a polysaccharide used according to the present invention is a cationic polysaccharide, notably cationic celluloses and galactomannan gums. Among the cationic polysaccharides, mention may be made more particularly of cellulose ether derivatives comprising quaternary ammonium groups, cationic cellulose copolymers or cellulose derivatives grafted with a water-soluble quaternary ammonium monomer and cationic galactomannan gums.

The cellulose ether derivatives comprising quaternary ammonium groups described in French patent 1 492 597, and in particular the polymers sold under the names “JR” (JR 400, JR 125 and JR 30M) or “LR” (LR 400 and LR 30M) by NALCO. These polymers are also defined in the CTFA dictionary as quaternary ammoniums of hydroxyethylcellulose that have reacted with an epoxide substituted with a trimethylammonium group.

Cationic cellulose copolymers or cellulose derivatives grafted with a water-soluble quaternary ammonium monomer are described in particular in U.S. Pat. No. 4,131,576, such as hydroxyalkyl celluloses, for instance hydroxymethyl-, hydroxyethyl- or hydroxypropylcelluloses grafted, in particular, with a methacryloylethyltrimethylammonium, methacrylamidopropyltrimethylammonium or dimethyldiallylammonium salt.

The commercial products corresponding to this definition are more particularly the products sold under the names Celquat L 200 and Celquat H 100 by National Starch.

The cationic galactomannan gums are described more particularly in patents U.S. Pat. No. 3,589,578 and 4 031 307, in particular guar gums containing trialkylammonium cationic groups. Guar gums modified with a salt (e.g. chloride) of 2,3-epoxypropyltrimethylammonium are used, for example.

Such products are sold in particular under the trade names Jaguar C13 S, Jaguar C 15, Jaguar C 17 or Jaguar C162 by the company Meyhall.

In a third embodiment, the composition according to the invention comprises at least one anionic polysaccharide as described above in combination with a cationic polysaccharide as described above.

Preferably in this third embodiment, said anionic polysaccharide is a polysaccharide containing fucose, galactose and galacturonic acid such as that sold under the name Fucogel Powder® (INCI name: Biosaccharide Gum-1), sold by the company SOLABIA and said cationic polysaccharide is a hydroxyethylcellulose crosslinked with epichlorohydrin and quaternized with trimethylamine (polyquaternium 10) such as that sold under the name JR 30M by the company DOW CHEMICAL.

More preferentially in this embodiment, said anionic polysaccharide is present in the composition according to the invention in an amount ranging from 0.1% to 1% by weight, relative to the total weight of the composition, preferably ranging from 0.2% to 0.4% by weight, relative to the total weight of the composition and said cationic polysaccharide is present in the composition according to the invention in an amount ranging from 0.1% to 1% by weight, relative to the total weight of the composition, preferably ranging from 0.3% to 0.6% by weight, relative to the total weight of the composition.

In order for the cosmetic or dermatological compositions of the invention to be more pleasant to use (softer on application, more nourishing, more emollient), it is possible to add an oily phase (or fatty phase) to the medium of these compositions.

This oily phase contains one or more oils, namely organic substances that are liquid at room temperature (20 to 25° C.) and atmospheric pressure (760 mmHg).

When it is present, the oily phase represents, preferably, from 0.1% to 50% of the total weight of the composition, better still from 1% to 30% and even better still from 5% to 20% of the total weight of the composition.

Mention may be made, as oils which can be used in the composition of the invention, for example, of:
hydrocarbon oils of animal origin, such as perhydrosqualene;
hydrocarbon oils of plant origin different from the two hydrogenated or non-hydrogenated polyoxyethylenated plant oils, such as liquid triglycerides of fatty acids comprising from 4 to 10 carbon atoms, such as triglycerides of heptanoic or octanoic acids, or else, for example, sunflower oil, corn oil, soybean oil, pumpkin oil, grapeseed oil, sesame oil, hazelnut oil, apricot oil, macadamia oil, arara oil, castor oil, avocado oil, caprylic/capric acid triglycerides, such as those sold by Stearinerie Dubois or those sold under the names Miglyol 810, 812 and 818 by Dynamit Nobel, jojoba oil or shea butter oil;
synthetic esters and ethers, especially of fatty acids, for instance the oils of formulae R1COOR2 and R1OR2 in which R1 represents the residue of a fatty acid containing from 8 to 29 carbon atoms and R2 represents a branched or unbranched hydrocarbon chain containing from 3 to 30 carbon atoms, for instance Purcellin oil, isononyl isononanoate, isopropyl myristate, 2-ethylhexyl palmitate, 2-octyldodecyl stearate, 2-octyldodecyl erucate or isostearyl isostearate; hydroxylated esters, for instance isostearyl lactate, octyl hydroxystearate, octyldodecyl hydroxystearate, diisostearyl malate or triisocetyl citrate; fatty alcohol heptanoates, octanoates or decanoates; polyol esters, for instance propylene glycol dioctanoate, neopentyl glycol diheptanoate and diethylene glycol diisononanoate; and pentaerythritol esters, for instance pentaerythrityl tetraisostearate;
linear or branched hydrocarbons of mineral or synthetic origin, such as volatile or non-volatile liquid paraffins, and derivatives thereof, petroleum jelly, polydecenes, and hydrogenated polyisobutene such as Parleam oil;
fatty alcohols containing from 8 to 26 carbon atoms, for instance cetyl alcohol, stearyl alcohol and a mixture thereof (cetylstearyl alcohol), octyldodecanol, 2-butyloctanol, 2-hexyldecanol, 2-undecylpentadecanol, oleyl alcohol or linoleyl alcohol;
partially hydrocarbon-based and/or silicone-based fluoro oils, such as those described in JP-A-2-295 912;
1 silicone oils, for instance volatile or non-volatile polymethylsiloxanes (PDMS) with a linear or cyclic silicone chain, which are liquid or pasty at room temperature, notably cyclopolydimethylsiloxanes (cyclomethicones) such as cyclohexasiloxane; polydimethylsiloxanes including alkyl, alkoxy or phenyl groups, which are pendent or at the end of a silicone chain, these groups containing from 2 to 24 carbon atoms; phenyl silicones, for instance phenyl trimethicones, phenyl dimethicones, phenyltrimethylsiloxydiphenylsiloxanes, diphenyl dimethicones, diphenylmethyldiphenyltrisiloxanes or 2-phenylethyl trimethylsiloxy silicates, and polymethylphenylsiloxanes;
mixtures thereof.

In the list of the abovementioned oils, the term “hydrocarbon oil” is understood to mean any oil predominantly comprising carbon and hydrogen atoms, and optionally ester, ether, fluoro, carboxylic acid and/or alcohol groups.

The other fatty substances that may be present in the oily phase are, for example, fatty acids containing from 8 to 30 carbon atoms, for instance stearic acid, lauric acid, palmitic acid and oleic acid; waxes, for instance lanolin, beeswax, carnauba wax or candelilla wax, paraffin waxes, lignite wax or microcrystalline waxes, ceresin or ozokerite, and synthetic waxes, for instance polyethylene waxes and Fischer-Tropsch waxes; gums such as silicone gums (dimethiconol); silicone resins such as trifluoromethyl-C₁-C₄-alkyl dimethicone and trifluoropropyl dimethicone; and silicone elastomers, for instance the products sold under the name KSG by the company Shin-Etsu, under the names Trefil, BY29 or EPSX by the company Dow Corning, or under the name Gransil by the company Grant Industries.

These fatty substances may be chosen in a varied manner by those skilled in the art in order to prepare a composition having the desired properties, for example in terms of consistency or texture.

In a known manner, the compositions of the invention may contain adjuvants that are common in the cosmetic and dermatological fields, other aqueous or lipophilic conventional gelling agents and/or thickeners; hydrophilic or lipophilic active agents; preservatives; antioxidants; fragrances; moisturizers; depigmenting agents; keratolytic agents; emulsifiers; vitamins; emollients; sequestrants; surfactants; polymers; acidifying or basifying agents; fillers; free-radical scavengers; ceramides; sunscreens (notably ultraviolet sunscreens); insect repellents; thinning agents; colorants (such as pigments, soluble dyes or nacres); bactericides; antidandruff agents. The amounts of these various adjuvants are those conventionally used in the fields under consideration.

Needless to say, those skilled in the art will take care to select the optional compound(s) to be added to the composition according to the invention such that the advantageous properties intrinsically associated with the composition in accordance with the invention are not, or are not substantially, adversely affected by the envisaged addition.

The compositions according to the invention may be in any form that is suitable for topical application, especially in the form of solutions of lotion or serum type, in the form of aqueous gels, aqueous-alcoholic gel, in the form of emulsions obtained by dispersing a fatty phase in an aqueous phase (O/W) or, conversely, (W/O) or in the form of multiple emulsions (O/W/O or W/O/W), of liquid or semi-liquid consistency such as milks, more or less rich creams, cream-gels or pastes. They can optionally be packaged as an aerosol and be provided in the form of a foam or of a spray. These compositions are prepared according to the usual methods.

The composition of the invention may have a viscosity suitable for the product that it is desired to obtain. This viscosity may range for example from 5 to 50 poise (0.5 to 5 Pa·s), more particularly from 10 to 20 poise, measured at room temperature (around 25° C.) with a Rheomat 180 using a no. 2 spindle, at 200 s−1.

When the composition is an emulsion, the oily phase contains one or more oils and optionally other fatty substances, as described above. The proportion of the oily phase of the emulsion may range for example from 5% to 80% by weight and preferably from 5% to 50% by weight relative to the total weight of the composition. The aqueous phase represents the balance to 100% by weight.

Advantageously, according to one particular embodiment of the invention, the emulsions prepared with the polymers according to the invention may be free of emulsifiers, while being stable upon storage, irrespective of the temperature (for example between 5° C. and) 50° ° C.

Thus, the compositions of the invention can be used as a care and/or hygiene product, such as creams for protecting, treating or caring for the face, for the hands or for the body, or protection or care body milks, or lotions, gels or foams for caring for the skin and mucous membranes or for cleansing or exfoliating the skin.

### Cosmetic Treatment Method

The present invention also relates to a cosmetic treatment method for caring for keratin materials, preferably the skin, comprising a step of applying the composition according to the invention to keratin materials, preferably the skin.

### Cosmetic Use

The present invention also relates to the cosmetic use of the composition according to the invention for caring for keratin materials, preferably the skin.

The composition according to the invention may be used in many cosmetic or dermatological applications where the presence of acidic active agents is useful, notably it may be used for treating or caring for the skin of the face and/or body, mucous membranes (lips) or scalp, for example to give the face a luminous and radiant complexion, a healthy glow, a smooth and younger appearance, to treat skin wrinkles and fine lines, to depigment the skin and notably to remove the marks of aging, and to eliminate comedones.

Throughout the description, including the claims, the expression “at least one” should be understood as being synonymous with “one or more”, unless otherwise specified.

The expressions “more than”, “between . . . and . . . ” and “ranging from . . . to . . . ” should be understood as being limits inclusive, unless otherwise specified.

The examples and figures that follow are presented as nonlimiting illustrations of the invention. The compounds are, depending on the case, cited as the chemical names or as the CTFA (International Cosmetic Ingredient Dictionary and Handbook) names.

### EXAMPLE

Comparative study of stability of the composition according to the invention, no. 1, compared to the compositions outside the invention, no. 2, no. 3 and no. 4.

The 4 gel-type compositions below were prepared.

TABLE 1

| | Composition | | | |
|---|---|---|---|---|
| | 1 according | Composition | Composition | Composition |
| | to the | 2 outside the | 3 outside the | 4 outside the |
| | invention | invention | invention | invention |
| INCI name (EU) | (m/m) | (m/m) | (m/m) | (m/m) |
| PH ADJUSTER | 0.86 | 0.86 | 0.86 | 0.86 |
| ACTIVE AGENT | 3.00 | 3.00 | 3.00 | 3.00 |
| PEG-60 HYDROGENATED | 0.30 | 1.00 | 0 | 1.00 |
| CASTOR OIL (EMULSOGEN | | | | |
| HCO 060, from CLARIANT) | | | | |
| SALICYLIC ACID (SALICYLIC | 1.50 | 1.50 | 1.50 | 1.50 |
| ACID from JQC (HUAYIN) | | | | |
| PHARMACEUTICAL) | | | | |
| FRAGRANCE | 0.10 | 0.10 | 0.10 | 0.10 |
| WATER-MISCIBLE ORGANIC | 20.00 | 20.00 | 20.00 | 20.00 |
| SOLVENT | | | | |
| POLYQUATERNIUM-10 | 0.45 | 0.45 | 0.45 | 0.45 |
| (UCARE POLYMER JR 30M | | | | |
| from AMERCHOL (DOW | | | | |
| CHEMICAL)) | | | | |
| PPG-26-BUTETH-26 (and) | 2.00 | 0.50 | 0.50 | 0 |
| PEG-40 HYDROGENATED | | | | |
| CASTOR OIL | | | | |
| (SOLUBILISANT LRI from | | | | |
| SENSIENT) | | | | |
| CAPRYLOYL SALICYLIC | 0.45 | 0.45 | 0.45 | 0.45 |
| ACID (MEXORYL SAB from | | | | |
| NOVEAL) | | | | |
| SURFACTANT | 0.50 | 0.50 | 0.50 | 0.50 |
| DIMETHYL ISOSORBIDE | 2.00 | 0 | 2.00 | 2.00 |
| (ARLASOLVE DMI-PC-LQ- | | | | |
| (AP) from CRODA) | | | | |
| GLYCOLIC ACID (GLYPURE | 5.00 | 5.00 | 5.00 | 5.00 |
| 70 from CHEMOURS) | | | | |
| SEQUESTRANT | 0.20 | 0.20 | 0.20 | 0.20 |
| BIOSACCHARIDE GUM-1 | 0.30 | 0.22 | 0.22 | 0.22 |
| (FUCOGEL POWDER from | | | | |
| SOLABIA) | | | | |
| CITRIC ACID (CITRIC ACID | 1.00 | 1.00 | 1.00 | 1.00 |
| MONOHYDRATE | | | | |
| GRANULAR from CITRIQUE | | | | |
| BELGE) | | | | |
| PRESERVATIVES | qs | qs | qs | qs |
| WATER | qs 100 | qs 100 | qs 100 | qs 100 |

### Production Process:

Homogenize the aqueous phase at room temperature under standardized stirring and verify good solubilization of all the raw materials.

Gel this aqueous phase then add the phase containing the AHAs/BHAS.

Lastly, add the pH adjuster.

The more or less clear macroscopic appearance and the presence/absence of crystals at room temperature, 4° ° C., 45° C. were evaluated for each of the above compositions by analysis with the naked eye.

TABLE 2

| Appearance | 4° C. | RT (25° C.) | 45° C. |
|---|---|---|---|
| Composition | Clear | Clear | Cloudy + border |
| no. 4 outside | | | |
| the invention | | | |
| Composition | Numerous | Clear | Clear |
| no. 3 outside | crystals | | |
| the invention | | | |
| Composition | A few crystals | Clear | Slightly cloudy + |
| no. 2 outside | | | border |
| the invention | | | |
| Composition | Clear | Clear | Slightly cloudy |
| no. 1 | | | and no border |
| according to | | | |
| the | | | |
| invention | | | |

Results: Composition no. 1 according to the invention remains stable under each of the conditions tested, unlike compositions no. 2, no. 3 and no. 4 outside the invention, for which the presence of crystals and/or a border, unacceptable for use by a consumer, is observed.

1. A composition comprising:
i. at least one hydroxy acid chosen from α-hydroxy acids, β-hydroxy acids, and mixtures thereof;
ii. at least one monoalkyl or dialkyl isosorbide, the alkyl group comprising from 1 to 5 carbon atoms;
iii. at least one polyethylene glycol-polypropylene glycol ether of formula (I): 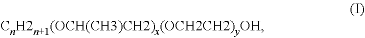
wherein:
a. n is an integer ranging from 1 to 24;
b. x is an integer ranging from 1 to 40;
c. y is an integer ranging from 1 to 40;
iv. at least one first hydrogenated or non-hydrogenated, polyoxyethylenated plant oil and at least one second hydrogenated or non-hydrogenated, polyoxyethylenated plant oil; said first and second polyoxyethylenated plant oils being different from one another;
v. a physiologically acceptable medium comprising at least water;
vi. said composition having an acidic pH.

2. The composition as claimed in claim 1, wherein the α-hydroxy acids are chosen from glycolic acid, citric acid, lactic acid, tartaric acid, malic acid, mandelic acid, and mixtures thereof.

3. The composition as claimed in claim 1, wherein the β-hydroxy acids are chosen from salicylic acid and the salicylic acid derivatives of the following formula (II): 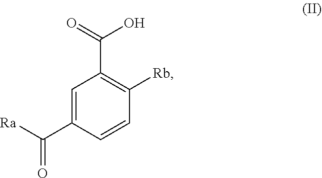
wherein:
i. the radical Ra denotes:
a. a linear, branched or cyclic, saturated aliphatic chain containing from 2 to 22 carbon atoms;
b. an unsaturated chain containing from 2 to 22 carbon atoms, containing one or more double bonds which may be conjugated;
c. an aromatic ring bonded to the carbonyl radical directly or by means of saturated or unsaturated aliphatic chains containing from 2 to 7 carbon atoms;
it being possible for said groups to be substituted with one or more identical or different substituents chosen from: (a) halogen atoms, (b) the trifluoromethyl group, (c) hydroxyl groups in free form or esterified with an acid containing from 1 to 6 carbon atoms, or (d) a carboxyl function in free form or esterified with a lower alcohol containing from 1 to 6 carbon atoms;
ii. Rb is a hydroxyl group;
and also the salts thereof derived from a mineral or organic base and mixtures thereof.

4. The composition as claimed in claim 1, wherein said monoalkyl or dialkyl isosorbide is dimethyl isosorbide.

5. The composition as claimed in claim 1, wherein said polyethylene glycol-polypropylene glycol ether of formula I is chosen from cetyl alcohol oxyethylenated with 20 oxyethylene units and oxypropylenated with 5 oxypropylene units, butyl alcohol oxyethylenated with 26 oxyethylene units and oxypropylenated with 26 oxypropylene units, decyltetradecanol oxyethylenated with 30 oxyethylene units and oxypropylenated with 6 oxypropylene units, lauryl alcohol oxyethylenated with 25 oxyethylene units and oxypropylenated with 25 oxypropylene units, and mixtures thereof.

6. The composition as claimed in claim 1, wherein said first hydrogenated or non-hydrogenated, polyoxyethylenated plant oil is a polyoxyethylenated plant oil comprising from 2 to 50 oxyethylene units and said second hydrogenated or non-hydrogenated, polyoxyethylenated plant oil is a polyoxyethylenated plant oil comprising from 51 to 100 oxyethylene units.

7. The composition as claimed in claim 1, wherein said hydroxy acid is present in the composition according to the invention in an amount ranging from 2% to 20% by weight relative to the total weight of the composition.

8. The composition as claimed in claim 1, wherein said monoalkyl or dialkyl isosorbide is present in the composition in an amount ranging from 0.5% to 10% by weight relative to the total weight of the composition.

9. The composition as claimed in claim 1, wherein said polyethylene glycol-polypropylene glycol ether of formula I is present in the composition in an amount ranging from 0.1% to 5% by weight, relative to the total weight of the composition.

10. The composition as claimed in claim 1, wherein said first hydrogenated or non-hydrogenated, polyoxyethylenated plant oil is present in the composition in an amount ranging from 0.1% to 2% by weight relative to the total weight of the composition, and said second hydrogenated or non-hydrogenated, polyoxyethylenated plant oil is present in the composition in an amount ranging from 0.05% to 0.5% by weight relative to the total weight of the composition.

11. A non-therapeutic cosmetic treatment method for caring for keratin materials comprising a step of applying the composition as claimed in claim 1 to said keratin materials.

12. The non-therapeutic method according to claim 11 wherein the keratin materials is the skin.

13. The non-therapeutic cosmetic treatment method according to claim 11, wherein the treatment comprises eliminating comedones.

14. The composition as claimed in claim 2, wherein the β-hydroxy acids are chosen from salicylic acid and the salicylic acid derivatives of the following formula (II): 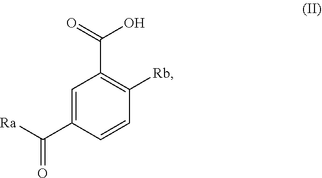
wherein:
i. the radical Ra denotes:
a. a linear, branched or cyclic, saturated aliphatic chain containing from 2 to 22 carbon atoms;
b. an unsaturated chain containing from 2 to 22 carbon atoms, containing one or more double bonds which may be conjugated;
c. an aromatic ring bonded to the carbonyl radical directly or by means of saturated or unsaturated aliphatic chains containing from 2 to 7 carbon atoms;
it being possible for said groups to be substituted with one or more identical or different substituents chosen from: (a) halogen atoms, (b) the trifluoromethyl group, (c) hydroxyl groups in free form or esterified with an acid containing from 1 to 6 carbon atoms, or (d) a carboxyl function in free form or esterified with a lower alcohol containing from 1 to 6 carbon atoms;
ii. Rb is a hydroxyl group;
and also the salts thereof derived from a mineral or organic base and mixtures thereof.

15. The composition as claimed in claim 2, wherein said monoalkyl or dialkyl isosorbide is dimethyl isosorbide.

16. The composition as claimed in claim 3, wherein said monoalkyl or dialkyl isosorbide is dimethyl isosorbide.

17. The composition as claimed in claim 2, wherein said polyethylene glycol-polypropylene glycol ether of formula I is chosen from cetyl alcohol oxyethylenated with 20 oxyethylene units and oxypropylenated with 5 oxypropylene units, butyl alcohol oxyethylenated with 26 oxyethylene units and oxypropylenated with 26 oxypropylene units, decyltetradecanol oxyethylenated with 30 oxyethylene units and oxypropylenated with 6 oxypropylene units, lauryl alcohol oxyethylenated with 25 oxyethylene units and oxypropylenated with 25 oxypropylene units, and mixtures thereof.

18. The composition as claimed in claim 3, wherein said polyethylene glycol-polypropylene glycol ether of formula I is chosen from cetyl alcohol oxyethylenated with 20 oxyethylene units and oxypropylenated with 5 oxypropylene units, butyl alcohol oxyethylenated with 26 oxyethylene units and oxypropylenated with 26 oxypropylene units, decyltetradecanol oxyethylenated with 30 oxyethylene units and oxypropylenated with 6 oxypropylene units, lauryl alcohol oxyethylenated with 25 oxyethylene units and oxypropylenated with 25 oxypropylene units, and mixtures thereof.

19. The composition as claimed in claim 4, wherein said polyethylene glycol-polypropylene glycol ether of formula I is chosen from cetyl alcohol oxyethylenated with 20 oxyethylene units and oxypropylenated with 5 oxypropylene units, butyl alcohol oxyethylenated with 26 oxyethylene units and oxypropylenated with 26 oxypropylene units, decyltetradecanol oxyethylenated with 30 oxyethylene units and oxypropylenated with 6 oxypropylene units, lauryl alcohol oxyethylenated with 25 oxyethylene units and oxypropylenated with 25 oxypropylene units, and mixtures thereof.

20. The composition as claimed in claim 2, wherein said first hydrogenated or non-hydrogenated, polyoxyethylenated plant oil is a polyoxyethylenated plant oil comprising from 2 to 50 oxyethylene units and said second hydrogenated or non-hydrogenated, polyoxyethylenated plant oil is a polyoxyethylenated plant oil comprising from 51 to 100 oxyethylene units.
